(19) United States
(12) Patent Application Publication
(10) Pub. No.: US 2012/0090471 A1
(43) Pub. Date: Apr. 19, 2012

(54) Scenting Device For HVAC Systems And Method Of Making A Scenting Device

(76) Inventor: Gary Robinson, Amherst, NY (US)

(21) Appl. No.: 13/272,423

(22) Filed: Oct. 13, 2011

Related U.S. Application Data
(60) Provisional application No. 61/392,805, filed on Oct. 13, 2010.

(51) Int. Cl. A61L 9/04; B05D 3/02; B05D 5/00

(52) U.S. Cl. 96/222; 427/244

(57) ABSTRACT

The invention may be embodied as a scenting device for attachment to a HVAC system. The scenting device may have a mesh substrate having a first face and a second face on opposing sides of the substrate, a perimeter, and openings. A layer of fragrant material may be adhered to the mesh substrate. A connector may be provided for attaching the mesh to the HVAC system.

### CROSS-REFERENCE TO RELATED APPLICATION

This application claims the benefit of priority to U.S. provisional patent application Ser. No. 61/392,805, filed on Oct. 13, 2010, which is incorporated herein by reference in its entirety.

### FIELD OF THE INVENTION

The present invention relates to improvements in air freshening devices such as those used in forced-air heating, ventilating and air conditioning (HVAC) systems.

### BACKGROUND OF THE INVENTION

Various air freshening or scenting devices have been proposed for imparting a desired fragrance to the air in homes and offices by inserting a fragrant material in the airstream of a HVAC system. Several devices of this type are currently available in the marketplace and generally consist of a substrate to which a fragrant material is attached. The substrate may be foam rubber and the fragrant material resides on its surface and/or in the pores of the foam. A desired scent is imparted to the air as the air contacts the fragrant material. Other devices place the fragrant material on card stock, and in use there is an opening in the card stock near the fragrant material to allow air to pass from one side of the card stock to another.

Although these devices have been commercially successful, they present certain important disadvantages. For example, foam rubber is often wet or greasy to the touch and is not pleasant to handle and install. Also, those devices that rely on air-impermeable substrates (such as card stock) restrict the airflow through the HVAC system, restrict the amount of air that is allowed to contact the fragrant material, and often require users to remove a portion of the substrate in order to create an opening through which air can pass.

### SUMMARY OF THE INVENTION

The invention may be embodied as a scenting device for attachment within a HVAC system, for example, to a HVAC filter. The scenting device may have a mesh substrate having a first face and a second face on opposing sides of the substrate, a perimeter, and openings. The mesh may be a semi-permeable barrier made of connected strands of material, such as flexible plastic. A layer of fragrant material may be adhered to the mesh substrate. A connector may be provided for attaching the mesh to the HVAC system, for example, to the HVAC filter.

The invention may also be embodied as a method of making a scenting device for attachment to a HVAC system through which air flows. In one such method, a sheet of mesh material, which has been sized to be received by the HVAC system, may be provided. The mesh substrate may have a first face and a second face on opposing sides. The mesh substrate may be placed on a backing substrate to facilitate manufacturing of the device. A layer of fragrant material may be disposed on the first face of the mesh substrate and allowed to flow to the second face so as to cover part of the first face and part of the second face. The fragrant material is allowed to set, and in doing so captures and adheres to a portion of the mesh so that the mesh can be used to support the fragrant material. The backing substrate keeps the fragrant material close to the mesh while the fragrant material is setting. Although the backing substrate may be attached to the fragrant material, which has been set, the backing substrate may be easily removable by hand from the fragrant material without harming the fragrant material after setting. To facilitate removal of the backing material, the backing substrate may be coated with or made from a material that makes it selectively removable from the set fragrant material.

To use the device, the backing substrate may be removed from the fragrant material, and the mesh may be supported in the HVAC system so as to expose the fragrant material to air that is moving through the HVAC system. When exposed to air moving through the HVAC system, the fragrant material evaporates from the mesh and travels away from the mesh to provide fragrance to the air.

### BRIEF DESCRIPTION OF THE DRAWINGS

For a fuller understanding of the nature and objects of the invention, reference should be made to the accompanying drawings and the subsequent description. Briefly, the drawings are:

FIG. 1 is a front view of a disposable furnace filter of a conventional HVAC system, showing a scenting device of the present invention mounted on the upstream face of the filter;

FIG. 2a is a front view of the scenting device shown in FIG. 1;

FIG. 2b is a front view of a different scenting device that is in keeping with the invention;

FIG. 3 is a side view of the scenting device shown in FIG. 1; and

FIG. 4 is a flow chart corresponding to a method of manufacturing the scenting device.

### DETAILED DESCRIPTION OF THE INVENTION

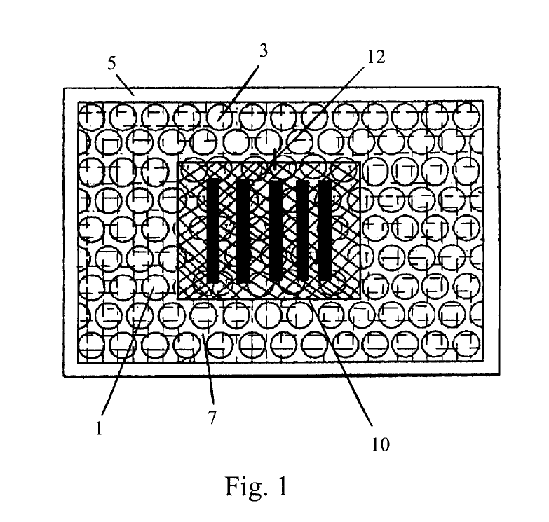

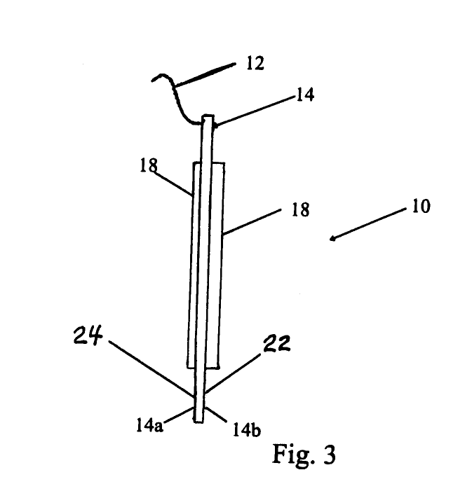

FIG. 1 shows a conventional disposable furnace filter 1 and is of the type that a consumer typically obtains from a hardware store or other retail outlet for use in a residence. A filter medium 3 is held within the rectangular frame 5 of the filter 1 and may include a reinforcing structure 7 to increase the rigidity of the filter assembly. A device 10 according to the present invention is shown secured to an HVAC system so that the device 10 resides against the upstream face of the filter. A simple connector, such as an S-hook 12, which is shown best in FIG. 3, may be used to attach the device 10 to the HVAC filter. Other connectors, such as pins, barbs, clips, clamps, tape, and adhesives could be utilized to hang the scenting device 10 from the filter 1. It should be understood that although a disposable air filter 1 is illustrated, the device of the present invention may be used with permanent HVAC filter assemblies. For example, the device 10 may be attached to an HVAC system so that the device 10 resides against an upstream face of a permanent HVAC filter assembly 1.

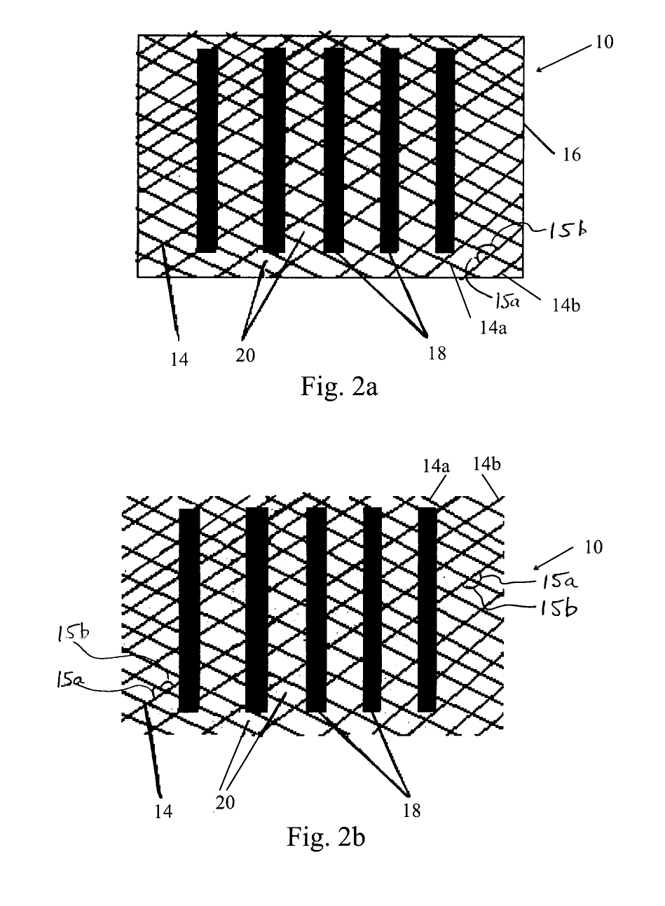

FIGS. 2a and 3 show an embodiment of a device 10 according to the invention. The scenting device 10 shown in FIG. 2a includes a mesh substrate 14. The mesh substrate 14 of FIG. 2a is shown having an approximately square shaped perimeter 16 but the invention is not limited to this shape. The perimeter material 16 need not be present, and this arrangement is depicted in FIG. 2b. The mesh 14 provides support for the fragrant material 18 and is preferably formed from a flexible material. Most preferably, the mesh 14 is a plastic mesh. Such a mesh 14 is easily trimmed (e.g. via scissors, knife or die) to a desired size. It should be appreciated, however, that other meshes, including those being rigid, or formed of a metal or other material, could also be used. The mesh may be made of connected strands 14a, 14b having a thickness of about 0.1 mm. The strands 14a, 14b can include a first set of substantially parallel strands 14a and a second set of substantially parallel strands 14b which intersect one another. As shown in FIG. 3, the first set 14a may be placed on top of the second set 14b so that the mesh 14 has a thickness approximately equal to twice the thickness of a single strand of material. The strands 14a, 14b may be angled relative to the perimeter 16 in a “cross-hatch” relationship, where the first set of strands 14a intersect the second set of strands 14b so that each of the included angles of intersection 15a, 15b are greater than 30 degrees. The mesh 14 may be used in a rectangular shape (such as square), or other shape as desired. In an embodiment of the invention, the overall size of the mesh 14 may be approximately six to seven inches square for use in typical residential HVAC systems.

The mesh 14 forms a plurality of openings 20 that provide passage through which the air to be scented flows in the HVAC system, thereby exposing the air to the fragrant material 18. Layers of fragrant material 18 may be disposed on the upstream face 22 of the mesh substrate. Alternatively, layers of fragrant material 18 may envelop parts of the mesh 14 so that the fragrant material 18 is disposed on both the upstream face 22 and the downstream face 24 of the substrate 14. In this manner, the portion of the mesh 14 that the fragrant material 18 is disposed on is encapsulated by the fragrant material 18 and this results in the fragrant material 18 being more securely attached to the mesh 14. The fragrant material 18 may be allocated on the mesh 14 so that more fragrant material 18 extends from the downstream face 24 more than the upstream face 22. But, it may be advantageous to allocate more fragrant material 18 to extend from that side of the mesh 14 that will face upstream (the upstream face 22) in the HVAC system because more fragrant material is expected to evaporate from that side of the mesh 14. FIG. 3 shows such an arrangement.

The layers of fragrant material 18 may be disposed so as to cover a plurality of the openings 20 in the mesh substrate 14. Preferably, the majority of openings 20 are not covered by the fragrant material 18 so that air flow through the HVAC system is not inhibited to an extent that would materially affect performance of the HVAC system, and to facilitate movement of the air across the downstream side of the fragrant material 18. In this manner, air is allowed to contact the fragrant material 18 on both the upstream 22 and downstream 24 sides of the scenting device 10. More particularly, air can flow around the fragrant material 18 of the upstream face 22 and pick-up fragrant material 18 located on the downstream face 24 of the mesh 14, thereby improving the emittance of fragrant material 18 into the air that is flowing through the HVAC system.

A suitable fragrant material 18 may be made by mixing a desired fragrant oil in a liquid petroleum distillate diluent such as Isopar M thereby producing a fragrant liquid. Increasing the amount of diluent increases the volume of fragrant liquid, which increases the time required for evaporation, thereby extending the time during which fragrance is imparted to the air. A gelling agent may be dissolved in the mixture to form the fragrant material, and at room temperature, the fragrant material 18 may be a high viscosity gel. Formulations may have a useful fragrant life of up to about two weeks.

In an embodiment of the invention, the fragrant material 18 has a gel composition that exhibits no flow (or very slow flow) at room-temperature so that the gel substantially maintains its shape during use. This allows the fragrant material 18 to be formed on the mesh 14 in the form of a layer having a thickness of at least about 1/16 inch, so that at least about 0.06 cubic inches of fragrant material 18 are applied for each square inch (including openings 20 in the mesh 14) of the mesh 14 that is covered. In one embodiment of the invention, approximately 22 grams of fragrant material 18 are applied to the mesh 14.

The fragrant material 18 may be applied by first heating it, preferably to approximately 230-250° F. Heating reduces the viscosity of the fragrant material 18 sufficiently so that the fragrant material 18 can be sprayed or flowed on the mesh in a variety of patterns. At these elevated temperatures, the fragrant material 18 flows and spreads to cover the mesh 14. As the fragrant material 18 cools, its viscosity increases and soon becomes a gel. Natural cooling may be augmented by a cooling medium in order to expedite formation of the gel. As the fragrant material 18 cools to room temperature, it adheres to the surface of the mesh 14 and its relatively high room-temperature viscosity allows it to remain in place on the mesh as a thick layer during subsequent processing and handling. Thus, even when the device 10 is oriented vertically, the layer of fragrant material does not run but maintains its geometric stability. The heating and subsequent cooling of the fragrant material may be controlled so as to produce a layer of fragrant gel that is at least about 1/16 inches thick.

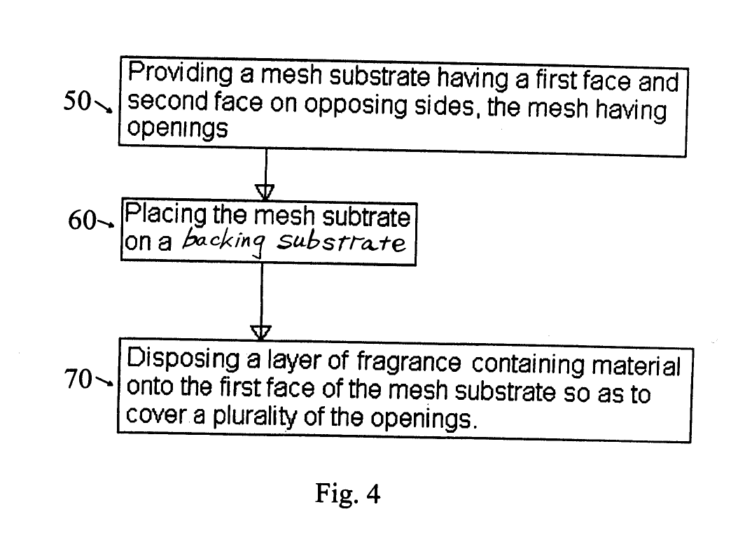

FIG. 4 illustrates one method of making a scenting device, such as that shown in FIG. 2a or FIG. 2b. First, a mesh having the desired overall shape is provided 50 from a suitable substrate material, such as a plastic mesh. Prior to placing the fragrant material, the mesh may be placed 60 on or near a backing substrate, which provides support for the fragrant material while it sets. Next, a layer of fragrant material is deposited 70 on the mesh, and the backing substrate holds the fragrant material in place around a portion of the mesh while the fragrant material sets. After the fragrant material is applied, it is preferably cooled to reduce the set-up time and minimize the extent to which the material flows upon application to the mesh. To accomplish rapid cooling, the device may be transported by conveyor through a cooling tunnel in which cooling air is directed over the material to decrease cooling time. After cooling, the device may be packaged for shipment. The backing substrate may be part of the shipping material, an in that event, the backing substrate would be removed prior to installing the mesh on an HVAC air filter.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A scenting device for attachment to a HVAC system, comprising
a mesh substrate having a first face and a second face on opposing sides of the substrate, and having openings;
a layer of fragrant material covering and adhered to a portion of the mesh substrate; and
a connector for attaching the mesh to the HVAC system.

2. The scenting device of claim 1, wherein the layer of fragrant material is a gel.

3. The scenting device of claim 1, wherein the layer of fragrant material envelops a portion of the mesh.

4. The scenting device of claim 1, wherein a majority of the openings are not covered by the layer of fragrant material.

5. The scenting device of claim 1, wherein at least about 0.06 cubic inches of fragrant material are applied for each square inch of the first face that is covered.

6. The scenting device of claim 1, wherein more of the fragrant material is disposed on the first face than the second face.

7. The scenting device of claim 1, further comprising a backing substrate removably attached to the layer of fragrant material.

8. The scenting device of claim 1, wherein the mesh substrate is made of a material that can be cut with scissors.

9. The scenting device of claim 1, wherein the perimeter of the mesh substrate is rectangular.

10. The scenting device of claim 9, wherein a side of the perimeter is between six and seven inches long.

11. A method of making a scenting device for attachment to a HVAC system through which air flows, comprising the steps of:
a.) providing a sheet of mesh material sized to be received by the HVAC system, the mesh substrate having a first face and second face on opposing sides, the mesh having openings;
b.) placing the mesh substrate on or near a backing substrate; and
c.) disposing a layer of fragrant material onto the first face of the mesh substrate.

12. The method of claim 11, wherein the layer of fragrant material covers a plurality of the openings.

13. The method of claim 11, further comprising disposing a second layer of fragrant material onto the first face of the mesh substrate.

14. The method of claim 11, further comprising heating the layer of fragrant material prior to step c.).

15. The method of claim 14, wherein the layer of fragrant material is heated to approximately 230-250° F.

16. The method of claim 11, wherein the layer of fragrant material is disposed on the backing substrate.

17. The method of claim 11, wherein the scenting device is exposed to cooling air in a cooling tunnel.

18. A scenting system, the system comprising:
a scenting device having:
(a) a mesh substrate having a first face and a second face on opposing sides of the substrate, and openings;
(b) a layer of fragrant material adhered to and encapsulating a portion of the mesh substrate; and
(c) a connector;
a HVAC filter for filtering air flowing through the HVAC system, the HVAC filter having an upstream face and a downstream face;
wherein the scenting device is attached via the connector so as to reside against the upstream face of the HVAC filter.

19. The scenting system of claim 18, wherein the layer of fragrant material is a gel.

20. The scenting system of claim 18, wherein a plurality of layers of fragrant material are adhered to the mesh substrate.

21. The scenting system of claim 18, wherein more of the fragrant material extends from the first face than the second face.

22. The scenting system of claim 18, further comprising a removable backing substrate attached to the layer of fragrant material.
